(19) United States
(12) Patent Application Publication
(10) Pub. No.: US 2011/0281956 A1
(43) Pub. Date: Nov. 17, 2011

(54) USE OF THYMUS CAPITATUS EXTRACT, SATUREJA HORTENSIS EXTRACT, OR CARVACROL FOR TREATING METABOLIC DISEASES

(75) Inventor: Tae Sun Park, Seoul (KR)

(73) Assignee: INDUSTRY-ACADEMIC COOPERATION FOUNDATION, YONSEI UNIVERSITY, Seoul (KR)

(21) Appl. No.: 13/190,020

(22) Filed: Jul. 25, 2011

Related U.S. Application Data
(63) Continuation of application No. PCT/KR2009/007304, filed on Dec. 8, 2009.

(30) Foreign Application Priority Data
Feb. 2, 2009 (KR) 10-2009-0008025

(51) Int. Cl. A61K 31/05; A61P 3/04; A61P 3/10; A61P 3/06; C07C 39/06; A61P 1/16

(52) U.S. Cl. 514/731; 568/781

(57) ABSTRACT

Disclosed is an use of Spanish oregano (Thymus capitatus) extract, summer savory (Satureja hortensis) extract or carvacrol for treating metabolic diseases. More specifically, the present disclosure relates to a composition containing Spanish oregano extract or summer savory extract, carvacrol, which is an aromatic oil component frequently found in mint, oregano, thyme, bergamot camphor tree or thyme thymol, as an active ingredient, for preventing and treating a metabolic disease selected from the group consisting of obesity, fatty liver, hyperlipidemia and diabetes, an use thereof, and a method of using the same. The disclosed composition, use and method are useful for weight loss, suppression of abdominal fat accumulation, treatment of fatty liver, reduction of cholesterol and free fatty acid in plasma and liver tissue, and improvement of blood glucose level and insulin resistance. Therefore, the composition can be effectively used to prevent and treat a metabolic disease selected from the group consisting of obesity, fatty liver, hyperlipidemia and diabetes.

### CROSS-REFERENCE TO RELATED APPLICATION

This application is a continuation of PCT/KR2009/007304 filed Dec. 8, 2009, which claims the benefit of Korean Application No. 10-2009-0008025 filed Feb. 2, 2009, the entire contents of which applications are incorporated herein by reference.

### TECHNICAL FIELD

This application claims priority to Korean Patent Application No. 10-2009-0008025, filed on Feb. 2, 2009, in the Korean Intellectual Property Office, the disclosure of which is incorporated herein by reference in its entirety.

The present disclosure relates to an use of Spanish oregano (Thymus capitatus) extract, summer savory (Satureja hortensis) extract or carvacrol for treating metabolic diseases. More specifically, the present disclosure relates to a composition containing carvacrol, which is an aromatic oil component frequently found in mint, oregano, thyme, bergamot, camphor tree or thyme thymol, Spanish oregano extract or summer savory extract, as an active ingredient, for preventing and treating a metabolic disease selected from the group consisting of obesity, fatty liver, hyperlipidemia and diabetes, an use thereof, and a method of using the same.

### BACKGROUND

With the rising of the standard of living and the prevalence of high-calorie diet, modern people suffer from various metabolic diseases.

Among them, obesity is a metabolic disease resulting from imbalance between calorie intake and consumption whereby excess body fat has accumulated to an abnormal extent (Kopelman and Stock, 1998). Men whose body fat takes up 25% or more of body weight and women whose body fat takes up 30% or more of body weight are considered obese. Clinically, people whose body mass index (BMI) is between 25.0 and 30.0 are classified as overweight, and those with BMI 30.0 or larger are defined as obese. There is a report that 70% of population in the West are overweight or obese (Colditz et al., 1995; Centers for Disease Control and Prevention, 2002). And, It is reported that 25% of Korean adults are overweight or obese (Korea Health Industry Development Institute, 2001). Prolonged obesity increases the likelihood of various diseases, including hypertension, increased blood cholesterol level, diabetes, kidney diseases, stroke, arteriosclerosis, fatty liver, arthritis, cancer, breathing difficulties during sleep, and so forth.

Among them, fatty liver refers to a condition where more fat is accumulated in the liver than normal level (−5%). With the improvement in nutritive conditions and increase in adult diseases, fatty liver patients are increasing. Most fatty liver patients are asymptomatic and appear healthy. Symptoms are various, including fatigue, lethargy, or pain in the right upper part of the belly. Medication is not generally given for fatty liver. Although metadoxine, betaine glucuronate, methionine, choline and lipotropic agents are used as complementary therapy, their effect is not medically proven.

Hyperlipidemia refers to the condition of abnormally elevated levels of fat components in the blood, causing inflammation on the vessel wall and leading to cardiovascular diseases. Like fatty liver, patients with hyperlipidemia are increasing with the recent improvement in nutritive conditions and increase in chronic diseases.

For treatment of hyperlipidemia, statin, ezetimibe, cholestyramine, niacin, fibrate, and omega-3 fatty acid such as eicosapentaenoic acid (EPA) and docosahexaenoic acid (DHA) are used.

Diabetes is a metabolic disease caused by insufficient secretion or function of insulin. It is characterized by high blood sugar, which results in various signs and symptoms, including glycosuria.

Diabetes is classified into type 1 diabetes and type 2 diabetes. Type 1 diabetes, also called “juvenile diabetes”, results from the body's failure to produce insulin. Type 2 diabetes results from insulin resistance, a condition in which cells fail to use insulin properly. Although it is thought that type 2 diabetes is caused primarily by environmental factors including westernized high-calorie, high-fat, high-protein diets, lack of exercise, stress, or the like, genetic defect may also be the cause of diabetes. In addition, it may be caused by pancreatic surgery, infections or drugs.

Carvacrol is a plant-derived aromatic oil present in mint, oregano, thyme, bergamot or camphor tree. It is reported that carvacrol has antibacterial and anticancer effects and inhibits bacterial growth by disrupting the bacterial cell membrane of Escheria coli, Bacillus cereus, or the like (Du W X, Olsen C E, Avena-Bustillos R J, McHugh T H, Levin C E, Friedman M (2008). “Storage Stability and Antibacterial Activity against Escherichia coli O157:H7 of Carvacrol in Edible Apple Films Made by Two Different Casting Methods”. J. Agric. Food Chem. 56: 3082). And, in an experiment where leiomyosarcoma cells isolated from rats cancer-induced with 3,4-benzopyrene were treated with carvacrol at 67 μM for 24 hours or at 90 μM for 48 hours, proliferation of cancer cells was inhibited and production of the final product of platelet aggregation thromboxane B₂ was decreased (Anticarcinogenic and antiplatelet effects of carvacrol. Exp. Oncol. 2006 Jun; 28 (2): 121-5).

Spanish oregano (Thymus capitatus L.) is a plant native to Mediterranean Europe. It has green leaves throughout the year and flowers bloom in July to September. The plant grows to about 0.25 m. Spanish oregano has been widely used primarily as spice. The essential oil of Spanish oregano is used in confectionery, spices, drinks and ice cream. The leaves harvested prior to blooming are used to prepare tea. For medical purposes, Spanish oregano has been used as insect repellent, insecticide and deodorant. Further, it is used in soap, mouth wash, fragrance, or the like.

Summer savory (Satureja hortensis L.) is distributed from Southeastern Europe to Western Asia. The leaves are used as spice or herbal tea. In addition, summer savory has been used for various medical purposes, particularly to help digestion, relieve stomach pain, discharge phlegm and treat arthritis. Besides, it is used for insect-repelling and anti-bacterial purposes.

### DISCLOSURE

### Techincal Problems

The inventors of the present disclosure have studied about new physiological activities of Spanish oregano (Thymus capitatus) extract, summer savory (Satureja hortensis) extract and carvacrol. They have found out that Spanish oregano extract, summer savory extract and carvacrol provide the effect of suppressing abdominal fat accumulation and preventing and treating obesity, fatty liver, hyperlipidemia and diabetes, and have developed a composition for preventing and treating a metabolic disease selected from the group consisting of obesity, fatty liver, hyperlipidemia and diabetes containing Spanish oregano extract, summer savory extract or carvacrol as an active ingredient, thereby completing the present invention.

Accordingly, the present disclosure is directed to providing an use of carvacrol for preventing and treating metabolic diseases.

The present disclosure is also directed to providing an use of Spanish oregano or summer savory extract for preventing and treating metabolic diseases.

### Technical Solutions

In one general aspect, the present disclosure provides a pharmaceutical composition for preventing and treating a metabolic disease selected from the group consisting of obesity, fatty liver, hyperlipidemia and diabetes, containing carvacrol or a pharmaceutically acceptable salt thereof as an active ingredient.

In another general aspect, the present disclosure provides an use of carvacrol or a pharmaceutically acceptable salt thereof for preparing an agent for preventing and treating a metabolic disease selected from the group consisting of obesity, fatty liver, hyperlipidemia and diabetes.

In another general aspect, the present disclosure provides a method for preventing and treating a metabolic disease selected from the group consisting of obesity, fatty liver, hyperlipidemia and diabetes, including administering an effective amount of carvacrol or a pharmaceutically acceptable salt thereof to a subject in need thereof.

In another general aspect, the present disclosure provides a food composition for preventing and treating a metabolic disease selected from the group consisting of obesity, fatty liver, hyperlipidemia and diabetes, containing carvacrol or a pharmaceutically acceptable salt thereof as an active ingredient.

In another general aspect, the present disclosure provides a pharmaceutical composition for preventing and treating a metabolic disease selected from the group consisting of obesity, fatty liver, hyperlipidemia and diabetes, containing Spanish oregano or summer savory extract as an active ingredient.

In another general aspect, the present disclosure provides an use of Spanish oregano or summer savory extract for preparing an agent for preventing and treating a metabolic disease selected from the group consisting of obesity, fatty liver, hyperlipidemia and diabetes.

In another general aspect, the present disclosure provides a method for preventing and treating a metabolic disease selected from the group consisting of obesity, fatty liver, hyperlipidemia and diabetes, including administering an effective amount of Spanish oregano or summer savory extract to a subject in need thereof.

In another general aspect, the present disclosure provides a food composition for preventing and treating a metabolic disease selected from the group consisting of obesity, fatty liver, hyperlipidemia and diabetes, containing Spanish oregano or summer savory extract as an active ingredient.

Hereinafter, exemplary embodiments will be described in detail with reference to the accompanying drawings.

The composition of the present disclosure comprising carvacrol or a pharmaceutically acceptable salt thereof as an active ingredient comprises carvacrol represented by Chemical Formula 1 or a salt thereof as an active ingredient and may be used to prevent and treat a metabolic disease selected from the group consisting of obesity, fatty liver, hyperlipidemia and diabetes:

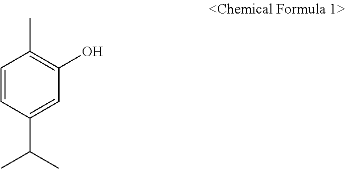

Carvacrol may be isolated and purified from natural materials or purchased commercially or prepared by chemical synthetic method which is well known in the art. It may be isolated from Carpenter's Weed (Achillea millefolium), Common Couch (Agropyron repens), Dill (Anethum graveolens), Chinese angelica (Angelica sinensis), Annual mugwort (Artemisia annua), Chamomile-leaved artemisia (Artemisia cina), Boldo (Boldea fragrans), Black Tea (Camellia sinensis), Caraway (Carum carvi), Bachelor's buttons (Chrysanthemum parthenium), Camphor (Cinnamomum camphora), American Dittany (Cunila origanoides), Akgiritotu (Dictamnus albus), Bitter root (Gentiana lutea), Commom Licorice (Glycyrrhiza glabra), Dokudami (Houttuynia cordata), Azob (Hyssopus officinalis), Black Walnut (Juglans nigra), Sabine (Juniperus sabina), Bay (Laurus nobilis), Lovage (Levisticum officinale), Gold-and-silver (Lonicera japonica), Barbados lilac (Melia azedarach), European pennyroyal (Mentha pulegium), Fish mint (Mentha spicata), Lemon bergamot (Monarda citriodora), Bee Balm (Monarda didyma), Horse mint (Monarda punctata), Common myrtle (Myrtus communis), Cat-mint (Nepeta cataria), Basil (Ocimum basilicum), Chinese-celery (Oenanthe javanica), Oregano (Origanum onites), Bible hyssop (Origanum syriacum), Greek oregano (Origanum vulgare hirtum), Black Pepper (Piper nigrum), Asian plantain (Plantago asiatica), Compass Plant (Rosmarinus officinalis), Garden Savory (Satureja hortensis), Savory (Satureja montana), peppertree (Schinus molle California), Honey Leaf (Stevia rebaudiana), Indian date (Tamarindus indica), Thymus capitatus, Breckland Thyme Plant (Thymus serpyllum), Common Thyme (Thymus vulgaris), Funk's thyme (Thymus×citriodorus), Spanish thyme (Thymus zygis), ajwain (Trachyspermum ammi), Common Valerian (Valeriana officinalis) or Corn (Zea Mays) which comprises carvacrol and preferably it may be isolated from Garden Savory or Thymus capitatus L. Method for isolation and purification may be performed by solvent extraction or chrolatography which is well known in the art. For example, extraction of carvacrol from the plants may be prepared using water C₁-C₆ alcohol such as ethanol, methanol, propanol, isopropanol, and butanol, organic solvent such as acetone, ether, chloroform, ethylacetate, methylenechloride, hexane, cyclohexane, petroliumether diethylether and benzene alone or in combination as well as above-mentioned solvent. In addition, isolation method by chromatography which is well known in the art, for example, by silicagel column chromatography, polar fraction may be prepared and the fraction could be isolated by or high-performance liquid chromatography (HPLC).

The composition of the present disclosure comprising Spanish oregano (Thymus capitatus) or summer savory (Satureja hortensis) extract as an active ingredient comprises Spanish oregano or summer savory extract as an active ingredient and may be used to prevent and treat a metabolic disease selected from the group consisting of obesity, fatty liver, hyperlipidemia and diabetes.

Spanish oregano is a plant native to Mediterranean Europe. It has been widely used primarily as spice and as insect repellent, insecticide and deodorant for medical purposes.

The leaves of summer savory are used as spice or herbal tea. In addition, summer savory has been used for various medical purposes, particularly to help digestion, relieve stomach pain, discharge phlegm and treat arthritis. Besides, it is used for insect-repelling and anti-bacterial purposes.

The Spanish oregano or summer savory extract may be prepared by a known solvent extraction method. As an extraction solvent, for example, water, a C₁-C₆ alcohol such as ethanol and methanol, an organic solvent such as acetone, ethyl acetate, n-hexane, diethyl ether and benzene, or a mixture thereof may be used. Specifically, a solvent selected from the group consisting of water, a C₁-C₆ alcohol and a mixture thereof may be used for the extraction.

Most specifically, the extraction solvent may be ethanol or a mixture of ethanol and water. The mixture of ethanol and water may comprise 80% of ethanol. The extraction may be performed by any common extraction methods, including cold extraction and hot extraction. When 80% ethanol is used for the extraction, the proportion of Spanish oregano or summer savory to ethanol is not particularly limited. For example, ethanol may be added to Spanish oregano or summer savory in an amount of 2 to 20 times based on weight. Specifically, ethanol may be added in an amount of 3 to 5 times based on Spanish oregano or summer savory in order to improve extraction efficiency. Extraction temperature is not particularly limited as long as the destruction of the extracted components can be avoided. Specifically, the extraction temperature may be 4° C. to 120° C. Most specifically, it may be 18° C. to 35° C. Extraction time may be different depending on the extraction temperature and the extraction solvent. Typically, the extraction is carried out for 0.5 to 48 hours, specifically 24 to 36 hours.

Spanish oregano or summer savory may be extracted after washing and then with or without drying. The drying may be performed by any method, including sun drying, shade drying, hot-air drying, and air drying. Furthermore, Spanish oregano or summer savory may be grounded to enhance the extraction efficiency.

The extract of the present disclosure includes not only one extracted using the above-described extraction solvent but also one purified according to a common method. For example, fractions obtained through a variety of further purification processes, including separation of precipitates using filter paper, separation using an ultrafiltration membrane with a predetermined molecular weight cutoff value, separation by various chromatography techniques (based on size, charge, hydrophobicity or affinity), bleaching, etc., are included in the scope of the Spanish oregano or summer savory extract of the present disclosure. Specifically, the extract of the present disclosure may be further separated through centrifugation and dried in vacuum.

In an example according to the present disclosure, in order to evaluate the effect of carvacrol on adipocyte differentiation and growth, mouse adipocytes (3T3-L1) were treated with carvacrol of various concentrations and the degree of adipocyte differentiation and cellular fat content were measured. As a result, carvacrol significantly reduced the differentiation of the preadipocytes in a concentration-dependent manner at concentrations 10 μM or higher, and also decreased the cellular fat content in a concentration-dependent manner (see FIG. 1).

In another example according to the present disclosure, the effect of carvacrol on abdominal fat accumulation, fatty liver and hyperlipidemia in high-fat diet mice was investigated. After 10 weeks of high-fat dieting, the carvacrol group showed significant reduction in epididymal fat weight (31%), perirenal fat weight (54%), mesenteric fat weight (51%), retroperitoneal fat weight (23%), and total abdominal fat weight (36%), as compared to the high-fat diet control group (HFD), revealing the excellent abdominal fat reduction effect of carvacrol. Also, the carvacrol group showed significant decrease in triglyceride level (30%), total cholesterol level (29%), LDL+VLDL cholesterol level (46%), atherogenic index (44%), and free fatty acid level (80%) in serum, as compared to the HFD group. In addition, the carvacrol group showed significant decrease in fasting blood sugar, insulin level, and insulin resistance index, as compared to the HFD group. Besides, the carvacrol group showed significant decrease in liver weight (25%), and triglyceride level (48%), cholesterol level (42%), and free fatty acid level (58%) in liver tissue, as compared to the HFD group. Accordingly, it can be seen that carvacrol has the effect of remarkably relieving fatty liver induced by high-fat diet, remarkably reducing cholesterols and free fatty acids in serum and liver tissue, and improving fasting blood sugar and insulin resistance.

In another example according to the present disclosure, the effect of Spanish oregano or summer savory extract on body weight increase, abdominal fat accumulation, fatty liver and hyperlipidemia in high-fat diet mice was investigated. As a result, after 8 weeks of high-fat dieting, the Spanish oregano or summer savory extract group showed significant decrease in body weight by 20% or 22%, and body weight increase by 38% or 42%, respectively, as compared to the HFD group. Also, epididymal fat weight, perirenal fat weight, mesenteric fat weight, and retroperitoneal fat weight decreased significantly. Total abdominal fat weight was decreased significantly by 34% for the Spanish oregano extract and by 36% for the summer savory extract. Accordingly, it can be seen that the Spanish oregano or summer savory extract has excellent obesity-treating and abdominal fat-reducing effect.

Also, the Spanish oregano group showed significant decrease in triglyceride level (28%) and total cholesterol level (24%) in serum, as compared to the HFD group. The summer savory group showed significant decrease in triglyceride level (21%) and total cholesterol level (21%) in serum.

The liver-to-body weight ratio was decreased significantly in the Spanish oregano group (31%) and in the summer savory group (25%), as compared to the HFD group.

The Spanish oregano group showed significant decrease in triglyceride level (37%) and cholesterol level (51%) in liver tissue, and the summer savory group significant decrease in triglyceride level (31%) and cholesterol level (38%) in liver tissue, as compared to the HFD group. Accordingly, it can be seen that the Spanish oregano or summer savory extract has excellent effect of reducing triglyceride and cholesterol levels in serum and liver tissue. Thus, they are effective in improving fatty liver and hyperlipidemia induced by high-fat diet.

Accordingly, the composition of the present disclosure comprising the Spanish oregano or summer savory extract, carvacrol or a salt thereof as an active ingredient may be provided as a pharmaceutical composition for preventing or treating a metabolic disease selected from the group consisting of obesity, fatty liver, hyperlipidemia and diabetes. The composition of the present disclosure may comprise the Spanish oregano or summer savory extract, carvacrol or a pharmaceutically acceptable salt thereof in an amount of 0.001-99.999 wt % as well as an excipient as balance.

In the present disclosure, carvacrol may be used as it is or in the form of a pharmaceutically acceptable salt. The expression “pharmaceutically acceptable” means that the salt is physiologically acceptable and normally causes no allergic or other similar adverse reactions when administered to human. The salt may be an acid addition salt formed from a pharmaceutically acceptable free acid. The free acid may be an organic acid or an inorganic acid. The organic acid includes citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, glutamic acid and aspartic acid, but is not limited thereto. And, the inorganic acid includes hydrochloric acid, bromic acid, sulfuric acid and phosphoric acid, but is not limited thereto.

The pharmaceutical composition according to the present disclosure comprising the Spanish oregano or summer savory extract, carvacrol or a pharmaceutically acceptable salt thereof as an active ingredient may comprise a pharmaceutically effective amount of the Spanish oregano or summer savory extract, carvacrol or a salt thereof alone or in combination with one or more pharmaceutically acceptable excipient. The expression “pharmaceutically effective amount” refers to an amount required to exhibiting a desired effect as compared to a negative control group. Specifically, it refers to an amount sufficient to treat or prevent a metabolic disease selected from the group consisting of obesity, fatty liver, hyperlipidemia and diabetes. The diabetes may be type 2 diabetes. The pharmaceutically effective amount of the Spanish oregano or summer savory extract, carvacrol or a pharmaceutically acceptable salt thereof may be 0.01-100 mg/day/kg body weight. However, the pharmaceutically effective amount may vary depending on the particular disease and severity thereof, age, body weight, physical conditions and sex of the patient, administration route, treatment period, or other various factors.

The expression “pharmaceutically acceptable” means that the composition is physiologically acceptable and is nontoxic without causing allergic or other similar adverse reactions, such as gastroenteric trouble or dizziness, when administered to human. The composition of the present disclosure may be prepared into various formulations depending on administration routes along with the pharmaceutically acceptable excipient according to methods known in the art. The administration route includes oral and parenteral routes. Examples of the parenteral administration route include transdermal, intranasal, intraabdominal, intramuscular, subcutaneous and intravenous routes.

For oral administration, the pharmaceutical composition of the present disclosure may be formulated into powder, granule, tablet, pill, sugar-coated tablet, capsule, liquid, gel, syrup, suspension, wafer, or the like together with an adequate excipient for oral administration according to a method known in the art. The excipient may include a filler such as a sugar including lactose, dextrose, sucrose, sorbitol, mannitol, xylitol, erythritol, maltitol, etc., a starch including corn starch, wheat starch, rice starch, potato starch, etc., a cellulose including cellulose, methyl cellulose, sodium carboxymethyl cellulose, hydroxypropyl methylcellulose, etc., gelatin, polyvinylpyrrolidone, or the like. Also, a disintegrant such as crosslinked polyvinylpyrrolidone, agar, alginic acid, sodium alginate, etc. may be added. In addition, the pharmaceutical composition may further include an anticoagulant, lubricant, wetting agent, fragrance, emulsifier, antiseptic, or the like.

For parenteral administration, the pharmaceutical composition of the present disclosure may be formulated into injection, transdermal system or nasal inhaler together with an adequate excipient for oral administration according to a method known in the art. The injection should be sterilized and be protected from contamination by microorganisms such as bacteria and fungi. The injection may include a solvent or a suspension medium such as water, ethanol, polyol (e.g., glycerol, propylene glycol, polyethylene glycol, etc.), a mixture thereof and/or vegetable oil as an excipient. More specifically, the excipient may be Hank's solution, Ringer's solution, phosphate buffered saline (PBS) containing triethanolamine, sterile water for injection, or isotonic solution such as 10% ethanol, 40% propylene glycol and 5% dextrose. The injection may further include various antibacterial and antifungal agents such as paraben, chlorobutanol, phenol, sorbic acid, thimerosal, etc. for protection from contamination by microorganisms. Further, the injection may include an isotonic agent such as sugar or sodium chloride in most cases. The transdermal administration system may be in the form of ointment, cream, lotion, gel, topical solution, paste, liniment, aerosol, and so forth. As used herein, “transdermal administration” refers to a delivery of the effective amount of the active ingredient included in the pharmaceutical composition topically into the skin.

These formulations are described in the Remington's Pharmaceutical Science, 15th Edition, 1975, Mack Publishing Company, Easton, Pa., which is well known in the pharmaceutical chemistry field.

The inhaler may be in the form of a pressurized pack or an aerosol spray delivered from a nebulizer using an adequate propellant compound, e.g. dichlorofluoromethane, trichlorofluoromethane, dichlorotetrafluoroethane, carbon dioxide, etc. for easier delivery. A pressurized aerosol may be equipped with a valve for delivering a unit dosage. A gelatin capsule or cartridge used in the inhaler may include a powder mixture of lactose, starch or other matrix.

Other pharmaceutically acceptable excipients may be consulted from Remington's Pharmaceutical Sciences, 19th ed., Mack Publishing Company, Easton, Pa., 1995.

The pharmaceutical composition of the present disclosure comprising the Spanish oregano or summer savory extract, carvacrol or a pharmaceutically acceptable salt thereof as an active ingredient may be administered in combination with a compound known to have an effect of preventing and treating obesity, fatty liver, hyperlipidemia and diabetes.

The present disclosure provides an use of carvacrol or a pharmaceutically acceptable salt thereof for preparing an agent for preventing and treating a metabolic disease selected from the group consisting of obesity, fatty liver, hyperlipidemia and diabetes.

The present disclosure also provides an use of the Spanish oregano or summer savory extract for preparing an agent for preventing and treating a metabolic disease selected from the group consisting of obesity, fatty liver, hyperlipidemia and diabetes.

The present disclosure also provides a method for preventing and treating a metabolic disease selected from the group consisting of obesity, fatty liver, hyperlipidemia and diabetes, comprising administering an effective amount of carvacrol or a pharmaceutically acceptable salt thereof to a subject in need thereof.

The present disclosure also provides a method for preventing and treating a metabolic disease selected from the group consisting of obesity, fatty liver, hyperlipidemia and diabetes, comprising administering an effective amount of Spanish oregano or summer savory extract to a subject in need thereof.

The carvacrol or a pharmaceutically acceptable salt thereof, the Spanish oregano or summer savory extract may be administered in an effective amount through various routes, including oral, transdermal, subcutaneous, intravenous or intramuscular routes. As used herein, the “effective amount” refers to an amount required to achieve a therapeutic effect for a metabolic disease selected from the group consisting of obesity, fatty liver, hyperlipidemia and diabetes when administered to a patient.

The “subject” may be an animal, specifically a mammal including human. Also, it may refer to a cell, tissue or organ derived from an animal. The subject may be patient in need of treatment.

The Spanish oregano or summer savory extract, carvacrol or a pharmaceutically acceptable salt thereof may be administered as it is or after being prepared into various formulations as described above. Specifically, they may be administered until the desired effect, i.e. the therapeutic effect for the metabolic disease selected from the group consisting of obesity, fatty liver, hyperlipidemia and diabetes, is achieved.

The Spanish oregano or summer savory extract, carvacrol or a pharmaceutically acceptable salt thereof may be administered via various routes according to methods known in the art. That is to say, they may be administered orally or parenterally, e.g. buccally, intramuscularly, intravenously, intradermally, intraarterially, intramedullarily, intradurally, intraabodominally, intranasally, intravaginally, rectally, sublingually or subcutaneously, or via gastrointestinal tracts, mucous membranes or respiratory organs.

Also, the Spanish oregano or summer savory extract, carvacrol or a salt thereof may be provided in the form of a food composition for improving a metabolic disease selected from the group consisting of obesity, fatty liver, hyperlipidemia and diabetes.

The food composition of the present disclosure may be in any form, including functional food, nutritional supplement, health food and food additives. The food composition may be prepared in various forms according to methods known in the art.

For example, the Spanish oregano or summer savory extract or carvacrol may be prepared into tea, juice or drink or into granule, capsule or powder as health food. Also, the Spanish oregano or summer savory extract or carvacrol may be mixed with a substance or active ingredient known to have an effect of improving a metabolic disease selected from the group consisting of obesity, fatty liver, hyperlipidemia and diabetes to prepare a composition.

And, as functional food, the Spanish oregano or summer savory extract or carvacrol may be added to drinks (including alcoholic drinks), fruits and processed products thereof (e.g., canned fruit, bottled fruit, jam, marmalade), fish, meat and processed products thereof (e.g., ham, sausage, corned beef), confectionery and noodles (e.g., udon, buckwheat noodles, instant noodles, spaghetti, macaroni), fruit juice, drinks, cookies, yeot, dairy products (e.g., butter, cheese), vegetable oils, margarine, vegetable proteins, retort foods, frozen foods, seasonings (e.g., soybean paste, soy sauce, sauce), or the like.

Also, the Spanish oregano or summer savory extract or carvacrol may be prepared into powder or concentrate to be used as food additive.

The above-mentioned skills for nucleotides and proteins were referenced in Maniatis et al., Molecular Cloning: A Laboratory Manual, Cold Spring Harbor Laboratory, Cold Spring Harbor, N.Y. (1982); Sambrook et al., Molecular Cloning: A Laboratory Manual, 2d Ed., Cold Spring Harbor Laboratory Press (1989); Deutscher, M., Guide to Protein Purification Methods. Enzymology, vol. 182. Academic Press. Inc., San Diego, Calif. (1990)

### Advantageous Effect

As described above, the present disclosure provides, for treatment of metabolic diseases, a composition for preventing and treating a metabolic disease selected from the group consisting of obesity, fatty liver, hyperlipidemia and diabetes, containing Spanish oregano, summer savory extract or carvacrol as an active ingredient, an use thereof, and a method of using the same. The composition, use and method of the present disclosure are useful for weight loss, suppression of abdominal fat accumulation, treatment of fatty liver, reduction of cholesterol and free fatty acid in serum and liver tissue, and improvement of blood glucose level and insulin resistance. Therefore, the composition can be effectively used to prevent and treat a metabolic disease selected from the group consisting of obesity, fatty liver, hyperlipidemia and diabetes.

### BRIEF DESCRIPTION OF THE DRAWINGS

FIG. 1 shows adipocyte (3T3L1) differentiation inhibition effect (A) and fat accumulation inhibition effect (B) of carvacrol. * or ** on the bars indicates significance according to Student's t-test (*: p<0.05, **: p<0.01).

FIG. 2 shows abdominal fat weight in various parts of mouse to which test diet has been fed (epididymal: epididymal fat, perirenal: perirenal fat, mesenteric: mesenteric fat, retroperitoneal: retroperitoneal fat). The characters on the bars indicate significance according to one-way ANOVA and Duncan's multiple range test.

### EXAMPLES

The examples and experiments will now be described. The following examples and experiments are for illustrative purposes only and not intended to limit the scope of this disclosure.

### Example 1

### Adipocyte Differentiation Inhibition Effect of Carvacrol

The effect of carvacrol (indole-3-carbinol) on adipocyte differentiation and growth was investigated using mouse adipocytes (3T3-L1). Preadipocytes 3T3L1 were placed on a 12-well plate and cultured to confluency in a 5% CO₂ incubator at 37° C. using DMEM containing 1% penicillin-streptomycin, 1% non-essential amino acids and 10% fetal bovine serum (FBS). The cultured 3T3-L1 cells were differentiated into adipocytes by culturing for 2 days in a medium containing 0.5 mM methylisobutylxanthine/1 μM dexamethasone/1 μg/mL insulin (DMI). The cells were differentiated into mature adipocytes by further culturing for 2 days in DMEM containing 1 μg/mL insulin. Subsequently, they were further cultured for 10 days while changing DMEM every other day to obtain fully differentiated adipocytes.

Starting from the day when DMI was added to the 3T3-L1 cells, carvacrol was treated at concentrations of 0.1, 1, 10, 50 and 100 μM every other day. Carvacrol was purchased from Sigma and used after dissolving in DMSO. For negative control, only DMSO was used. After the culturing for a total of 14 days, when the cells were fully differentiated, the medium was removed and the fats contained in the differentiated adipocytes were stained. For this, the cells were washed 2 times with phosphate buffered saline (PBS), immobilized in 10% buffered neutral formalin for 1 hour, washed again once with PBS, stained for 1 hour by adding 1 mL of Oil Red O, which specifically stains fats red, to the 12-well plate, and then washed 2 times with distilled water.

To measure the triglyceride level of the differentiated 3T3-L1 cells, the stained cells were dissolved in 1 mL of isobutanol and OD value was measured at 600 nm.

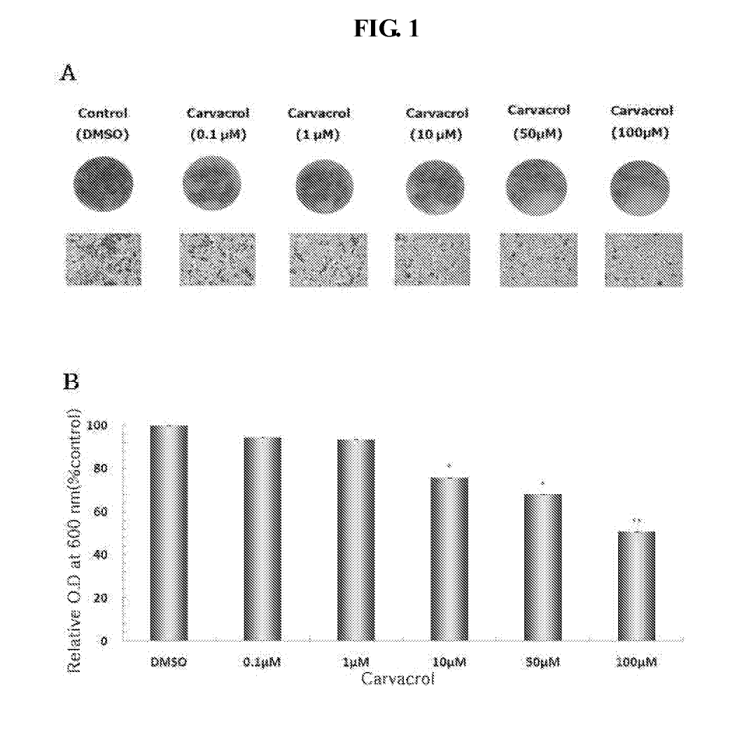

As seen from FIG. 1, carvacrol decreased the differentiation of the preadipocytes 3T3-L1 in a concentration-dependent manner at concentrations 10 μM or higher (A). Measurement of fats stained with Oil Red O also revealed the decrease in OD value in a concentration-dependent manner (B).

### Example 2

### Effect of Carvacrol On Suppression of Fat Accumulation And Improvement of Blood Glucose And Insulin Resistance

### <2-1>Preparation of Test Diets And Rearing of Test Animals

High-fat diet (HFD: 40% fat calorie, 17 g lard+3% corn oil/100 g diet) was used to induce obesity. Carvacrol-containing diet had the same composition as the HFD except for containing 0.1% carvacrol. Normal diet (ND) was prepared according to AIN-76 rodent diet (see Table 1).

TABLE 1
Composition of test diets

| | ND | HFD | Carvacrol | TCD | SHD |
|---|---|---|---|---|---|
| | (g/kg | (g/kg | (g/kg | (g/kg | (g/kg |
| | diet) | diet) | diet) | diet) | diet) |
| Casein | 200 | 200 | 200 | 200 | 200 |
| D/L-Methionine | 3 | 3 | 3 | 3 | 3 |
| Corn starch | 150 | 111 | 110 | 110 | 110 |
| Sucrose | 500 | 370 | 370 | 370 | 370 |
| Cellulose | 50 | 50 | 50 | 50 | 50 |
| Corn oil | 50 | 30 | 30 | 30 | 30 |
| Lard | — | 170 | 170 | 170 | 170 |
| Vitamin complex | 10 | 12 | 12 | 12 | 12 |
| Mineral complex | 35 | 42 | 42 | 42 | 42 |
| Choline bitartrate | 2 | 2 | 2 | 2 | 2 |
| Cholesterol | — | 10 | 10 | 10 | 10 |
| tert-Butylhydro- | 0.01 | 0.04 | 0.04 | 0.04 | 0.04 |
| quinone | | | | | |
| Carvacrol | — | — | 1.00 | — | — |
| Thymus capitatus | — | — | — | 1.00 | — |
| extract | | | | | |
| Satureja hortensis | — | — | — | — | 1.00 |
| extract | | | | | |
| Total (g) | 1,000 | 1,000 | 1,000 | 1,000 | 1,000 |
| Fat (% calorie) | 11.5 | 39.0 | 39.0 | 39.0 | 39.0 |
| Total calorie | 16,439 | 19,315 | 19,315 | 19,315 | 19,315 |
| (kJ/kg diet) | | | | | |

ND: normal diet
HFD: control high-fat diet
Carvacrol: carvacrol-containing diet
TCD: Spanish oregano (Thymus capitatus)-containing diet
SHD: summer savory (Satureja hortensis)-containing diet

5-week-old male C57BL/6J mice were accustomed to the laboratory environment for a week while feeding solid feed. They were randomly divided into the ND, HFD and carvacrol groups according to the randomized block design and reared for 10 weeks. Diet was provided between 10 and 11 a.m. every day along with water. Food intake was measured every day, and body weight was measured every 3 days. In order to avoid transient body weight increase due to feed intake, body weight was measured 2 hours after removing the feedbox.

### <2-2>Effect of Suppressing Abdominal Fat Accumulation

After fasting the test animal for at least 12 hours and anesthetizing with diethyl ether, blood, liver and abdominal fat tissue (epididymal, perirenal, mesenteric and retroperitoneal fat) were taken and weighed after washing with 0.1 M PBS (pH 7.4). The blood taken from the abdominal aorta was centrifuged at 1000×g for 15 minutes to isolate the serum.

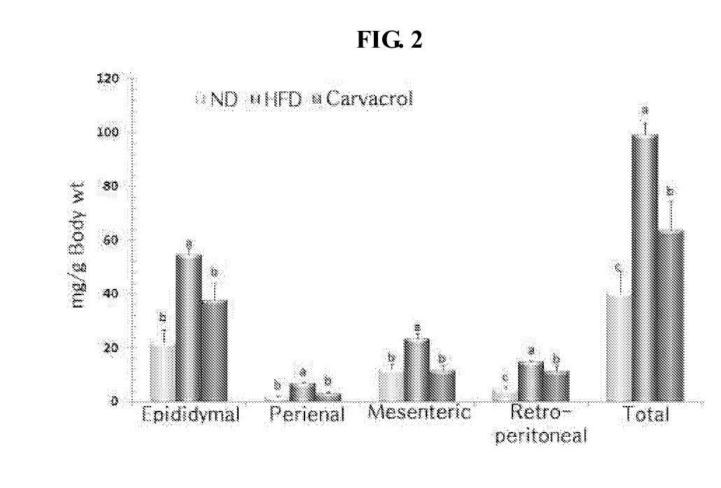

As seen from FIG. 2, the carvacrol group showed significant decrease in epididymal fat weight (31%), perirenal fat weight (54%), mesenteric fat weight (51%), retroperitoneal fat weight (23%) and total abdominal fat weight (36%) as compared to the control group (HFD) (p<0.001). Accordingly, it can be seen that carvacrol has an excellent effect of reducing abdominal fat.

### <2-3>Effect of Preventing And Treating Hyperlipidemia, Fatty Liver And Diabetes

Total cholesterol level, triglyceride level, glucose level and insulin level in plasma and lipid components in liver tissue were measured as follows. Total cholesterol level, triglyceride level and glucose level in plasma were measured 2 times using a commercially available kit (Bio Clinical System), and insulin level was measured by ELISA using a mouse Insulin kit (Shibayali, Japan). Lipid components were extracted from liver tissue according to Folch et al.'s method (Folch J, Lees M, Sloane Stanley G H. A simple method for the isolation and purification of total lipids from animal tissues. J. Biol. Chem. 1957; 226: 497-509) as follows. After adding 1 mL of distilled water to 0.25 g of liver tissue, the liver tissue was homogenized using a Polytron homogenizer (IKA-WERKE GmbH & Co., Ultra-Turrax, Staufen, Germany). After adding 5 mL of a chloroform/methanol mixture solution (2:1, v/v) to the homogenate and mixing well, centrifugation was performed at 1000×g for 10 minutes. Then, after separating the pellets and adding 2 mL of the chloroform/methanol mixture solution (2: 1, v/v) again to the supernatant, the same procedure was repeated to completely isolate the lipid components. After adding 3 mL of a chloroform/methanol/0.05% CaCl₂ mixture solution (3:48:47, v/v/v) to thus obtained pellets and mixing for 1 minute, centrifugation was performed at 1000×g for 10 minutes. Finally, the resulting pellets were completely dried using nitrogen gas and the dried lipids were dissolved in 1 mL of methanol for analysis of the lipid components. Triglyceride level of the liver tissue was measured as in the plasma analysis using the same commercially available kit (Bio Clinical System).

As a result, the carvacrol group showed significant decrease in triglyceride level (30%), total cholesterol level (29%), LDL+VLDL cholesterol level (46%), atherogenic index (44%) and free fatty acid level (80%) in the plasma as compared to the high-fat diet control group (HFD), as described in Table 2. Also, the carvacrol group showed significant decrease in fasting blood sugar, insulin level and insulin resistance index as compared to the HFD group. In Table 2, the characters a, b and c indicate significance according to one-way ANOVA and Duncan's multiple range test (p<0.001). The atherogenic index was calculated by (total cholesterol−HDL cholesterol)/HDL cholesterol, and the insulin resistance index was calculated by 10⁻³ pmol insulin×mmol glucose×L⁻².

TABLE 2
Blood lipid level and insulin resistance-related biological
indices in mice to which carvacrol was administered

| | Normal | High-fat | |
|---|---|---|---|
| | diet | diet control | |
| | (ND) | (HFD) | Carvacrol |
| Triglyceride (mmol/L) | 0.60 ± 0.09^{c} | 1.41 ± 0.12^{a} | 0.98 ± 0.14^{b} |
| Total cholesterol | 2.02 ± 0.06^{c} | 3.83 ± 0.32^{a} | 2.73 ± 0.30^{b} |
| (mmol/L) | | | |
| HDL cholesterol | 1.33 ± 0.09^{b} | 1.78 ± 0.07^{a} | 1.68 ± 0.16^{ab} |
| (mmol/L) | | | |
| LDL + VLDL | 0.69 ± 0.15^{b} | 2.05 ± 0.25^{a} | 1.11 ± 0.05^{b} |
| cholesterol (mmol/L) | | | |
| Atherogenic index | 0.59 ± 0.20^{b} | 1.02 ± 0.18^{a} | 0.57 ± 0.18^{b} |
| Free fatty acid (uEq/L) | 573 ± 36^{b} | 1417 ± 128^{a} | 280 ± 35^{c} |
| Glucose (mmol/L) | 6.16 ± 0.95^{b} | 8.49 ± 0.53^{a} | 6.83 ± 0.31^{b} |
| Insulin (ng/mL) | 0.51 ± 0.06^{b} | 0.76 ± 0.10^{a} | 0.58 ± 0.06^{b} |
| Insulin resistance index | 0.54 ± 0.07^{b} | 1.10 ± 0.10^{a} | 0.68 ± 0.08^{b} |

And, as seen from Table 3, the carvacrol group showed significant decrease in liver-to-body weight ratio (25%) as compared to the high-fat diet control group. The carvacrol group showed significant decrease in triglyceride level (48%), cholesterol level (42%) and free fatty acid level (58%) in the liver tissue as compared to the high-fat diet control group. Accordingly, it can be seen that carvacrol has an effect of remarkably relieving fatty liver induced by high-fat diet and remarkably improving cholesterol and free fatty acid levels in the liver tissue. In Table 3, the characters a, b and c indicate significance according to one-way ANOVA and Duncan's multiple range test (p<0.05).

TABLE 3
Liver weight and lipid level in liver tissue in
mice to which carvacrol was administered

| | Normal | High-fat | |
|---|---|---|---|
| | diet | diet control | |
| | (ND) | (HFD) | Carvacrol |
| Liver weight | 3.2 ± 0.2^{c} | 5.5 ± 0.3^{a} | 4.1 ± 0.1^{b} |
| (g/100 g body wt) | | | |
| Triglyceride (μmol/g) | 20.4 ± 1.21^{b} | 35.1 ± 1.22^{a} | 22.0 ± 6.0^{b} |
| Cholesterol (μmol/g) | 23.3 ± 2.31^{c} | 76.5 ± 2.51^{a} | 45.4 ± 5.5^{b} |
| Free fatty acid (uEq/g) | 8.6 ± 1.13^{b} | 24.3 ± 0.86^{a} | 10.3 ± 0.73^{b} |

### Example 3

### Effect of Summer Savory Or Spanish Oregano Extract In Reducing Body Weight And Abdominal Fat Weight And Preventing And Treating Hyperlipidemia And Fatty Liver

### <3-1>Preparation of Extract

500 g of dried leaves of Spanish oregano or summer savory were ground into powder and extracted in 2 L of 80% ethanol for 36 hours at 30° C. The resulting extract was filtered and centrifuged at 14,000 rpm for 10 minutes. The resulting supernatant was dried in vacuum to obtain the extract in powder form.

The final yield of the ethanol extracts of Spanish oregano leaves and summer savory leaves was 8.11% (w/w) and 7.69% (w/w), respectively.

### <3-2>Preparation of Test Diets And Rearing of Test Animals

The same high-fat diet (HFD, 40% fat calorie, 17 g lard+3% corn oil/100 g diet) as that used in Example 2-1 was used to induce obesity. Spanish oregano extract-containing diet (TCD) or summer savory extract-containing diet (SHD) had the same composition as the HFD except for containing 0.1% Spanish oregano or summer savory extract. Normal diet (ND) was prepared according to AIN-76 rodent diet (see Table 1).

5-week-old male C57BL/6J mice were accustomed to the laboratory environment for a week while feeding solid feed. They were randomly divided into the ND, HFD and TCD and SHD groups according to the randomized block design and reared for 8 weeks. Diet was provided between 10 and 11 a.m. every day along with water. Diet intake was measured every day, and body weight was measured every 3 days. In order to avoid transient body weight increase due to feed intake, body weight was measured 2 hours after removing the feedbox.

### <3-3>Measurement of Body Weight And Abdominal Fat-Pad Weight

Body weight increase was calculated from the body weight measured in Example 3-2.

After fasting the test animal for at least 12 hours and anesthetizing with diethyl ether, blood, liver and abdominal fat tissue (epididymal, perirenal, mesenteric and retroperitoneal fat) were taken and weighed after washing with 0.1 M PBS (pH 7.4). The blood taken from the abdominal aorta was centrifuged at 1000×g for 15 minutes to isolate the serum.

The Spanish oregano extract group showed significant decrease in body weight (20%) and body weight increase (38%) as compared to the high-fat diet control group. And, the Spanish oregano extract group showed significant decrease in epididymal fat weight, perirenal fat weight, mesenteric fat weight and retroperitoneal fat weight as compared to the control group. Total abdominal fat weight was significantly decreased by 34% as compared to the high-fat diet control group (p<0.05). Accordingly, it can be seen that Spanish oregano extract has an excellent effect of reducing body weight and abdominal fat (see Table 4).

The summer savory extract group showed significant decrease in body weight (22%) and body weight increase (42%) as compared to the high-fat diet control group. And, the summer savory extract group showed significant decrease in epididymal fat weight, perirenal fat weight, mesenteric fat weight and retroperitoneal fat weight as compared to the control group. Total abdominal fat weight was significantly decreased by 36% as compared to the high-fat diet control group (p<0.05). Accordingly, it can be seen that summer savory extract has an excellent effect of reducing body weight and abdominal fat (see Table 4).

TABLE 4
Body weight, body weight increase and abdominal fat weight of mice

| | ND | HFD | TCD | SHD |
|---|---|---|---|---|
| Body weight, | 29.7 ± 0.5^{c} | 42.4 ± 0.9^{a} | 33.6 ± 0.6^{b} | 32.7 ± 0.7^{b} |
| final (g) | | | | |
| Body weight | 10.7 ± 0.4^{c} | 23.4 ± 0.7^{a} | 14.5 ± 0.6^{b} | 13.5 ± 0.9^{b} |
| increase | | | | |
| (g/8 weeks) | | | | |
| Epididymal fat | 28.0 ± 2.14^{c} | 51.9 ± 5.86^{a} | 32.0 ± 3.55^{b} | 30.0 ± 3.99^{b} |
| weight (mg/g | | | | |
| body weight) | | | | |
| Retroperitoneal | 11.3 ± 1.24^{b} | 18.3 ± 1.91^{a} | 12.1 ± 1.08^{b} | 13.1 ± 1.02^{b} |
| fat weight | | | | |
| (mg/g body | | | | |
| weight) | | | | |
| Mesenteric fat | 10.1 ± 0.91^{c} | 17.3 ± 1.23^{a} | 12.7 ± 1.26^{b} | 12.7 ± 1.11^{b} |
| weight (mg/g | | | | |
| body weight) | | | | |
| Perirenal fat | 3.2 ± 0.41^{c} | 5.0 ± 0.72^{a} | 3.8 ± 0.29^{a} | 3.6 ± 0.27^{a} |
| weight (mg/g | | | | |
| body weight) | | | | |
| Total abdominal | 52.6 ± 6.3^{c} | 92.5 ± 10.5^{a} | 60.6 ± 3.31^{a} | 59.6 ± 2.94^{a} |
| fat weight | | | | |
| (mg/g body | | | | |
| weight) | | | | |

ND: normal diet
HFD: control high-fat diet
TCD: Spanish oregano (Thymus capitatus)-containing diet
SHD: summer savory (Satureja hortensis)-containing diet

### <3-4>Improvement of Hyperlipidemia And Fatty Liver

Total cholesterol level and triglyceride level in plasma and lipid components in liver tissue of the test animals were measured. Total cholesterol level and triglyceride level in plasma were measured 2 times in the same manner as in Example 2-3 using a commercially available kit (Bio Clinical System).

Lipid components were extracted from liver tissue according to Folch et al.'s method and measured in the same manner as in Example 2-3.

The Spanish oregano extract group showed significant decrease in plasma triglyceride level (28%) and total cholesterol level (24%) as compared to the high-fat diet control group (p<0.05). The Spanish oregano extract group showed significant decrease in liver-to-body weight ratio (31%) as compared to the high-fat diet control group (p<0.05). Also, the Spanish oregano extract group showed significant decrease in triglyceride level (37%) and cholesterol level (51%) in the liver tissue as compared to the high-fat diet control group (p<0.05) (see Table 5). Accordingly, it can be seen that the Spanish oregano extract has an effect of remarkably relieving fatty liver induced by high-fat diet and remarkably improving blood triglyceride and cholesterol level.

The summer savory extract group showed significant decrease in blood triglyceride level and total cholesterol level (21%) as compared to the high-fat diet control group (p<0.05). The summer savory extract group showed significant decrease in liver-to-body weight ratio (25%) as compared to the high-fat diet control group (p<0.05). Also, the summer savory extract group showed significant decrease in triglyceride level (31%) and cholesterol level (38%) in the liver tissue as compared to the high-fat diet control group (p<0.05) (see Table 5). Accordingly, it can be seen that the summer savory extract has an effect of remarkably improving fatty liver and hyperlipidemia induced by high-fat diet.

In Table 5, the characters a, b and c indicate significance according to one-way ANOVA and Duncan's multiple range test (p<0.05).

TABLE 5
Lipid level in blood and liver tissue of mice

| | ND | HFD | TCD | SHD |
|---|---|---|---|---|
| Plasma triglyc- | 0.60 ± 0.09^{c} | 1.51 ± 0.12^{a} | 1.08 ± 0.10^{b} | 1.19 ± 0.14^{b} |
| eride (mmol/L) | | | | |
| Plasma total | 2.12 ± 0.06^{c} | 3.33 ± 0.22^{a} | 2.53 ± 0.20^{b} | 2.63 ± 0.31^{b} |
| cholesterol | | | | |
| (mmol/L) | | | | |
| Liver weight | 2.8 ± 0.2^{c} | 4.8 ± 0.3^{a} | 3.3 ± 0.11^{b} | 3.6 ± 0.19^{b} |
| (g/100 g | | | | |
| body wt) | | | | |
| Liver tissue | 19.4 ± 1.21^{b} | 33.1 ± 1.22^{a} | 21.0 ± 4.5^{b} | 23.0 ± 5.1^{b} |
| triglyceride | | | | |
| (μmol/g) | | | | |
| Liver tissue | 23.3 ± 2.31^{c} | 65.5 ± 2.51^{a} | 32.4 ± 4.8^{b} | 40.4 ± 3.9^{b} |
| cholesterol | | | | |
| (μmol/g) | | | | |

ND: normal diet
HFD: control high-fat diet
TCD: Spanish oregano (Thymus capitatus)-containing diet
SHD: summer savory (Satureja hortensis)-containing diet

### Example of Preparation 1

### Preparation of Powder

After mixing the below ingredients and filling in an airtight pouch and they were prepared into powder according to a well known method.

carvacrol 50 mg

crystallized cellulose 2 g

### Example of Preparation 2

### Preparation of Tablet I

After mixing the below ingredients and performing direct compression and they were prepared into tablet according to a well known method.

carvacrol 50 mg

crystallized cellulosed 400 mg

magnesium stearate 5 mg

### Example of Preparation 3

### Preparation of Tablet II

After mixing the below ingredients and performing direct compression and they were prepared into tablet according to a well known method.

Spanish oregano extract 400 mg

crystallized cellulose 100 mg

magnesium stearate 5 mg

### Example of Preparation 4

### Preparation of Tablet III

Mixing Spiturina 55% (w/w), guagum enzyme extract 10% (w/w), Vitamin B₁ hydrochloride 0.01% (w/w), Vitamin B₆ hydrochloride 0.01% (w/w), DL-methionin 0.23% (w/w), magnesium stearate 0.7% (w/w), lactose 22.2% (w/w), corn starch 1.85% (w/w) and extract of Garden savory 10% (w/w), then performing direct compression and they were prepared into tablet according to a well known method.

### Example of Preparation 5

### Preparation of Capsule I

After mixing the below ingredients and filling into gelatin capsule and they were prepared into capsule according to a well known method.

carvacrol 30 mg

whey protein 100 mg

crystallized cellulose 400 mg

magnesium stearate 6 mg

### Example of Preparation 6

### Preparation of Capsule II

After mixing the below ingredients and filling into gelatin capsule and they were prepared into capsule according to a well known method.

carvacrol 300 mg

corn starch 100 mg

crystallized cellulose 100 mg

magnesium stearate 5 mg

### Example of Preparation 7

### Injection

Active ingredient is dissolved into distilled water for injection according to a well known method, and adjust pH to 7.5 and whole the below ingredient were dissolved in distilled water for injection. Then filled in 2 ml of ampoule, sterilized and injection were prepared.

carvacrol 100 mg

suitable amount of distilled water for injection

suitable amount of pH adjusting agent

### Example of Preparation 8

### Seonsik

Alpharized and dried by well known method brown rice, barley, sweet rice, Job's tears were ground with granule size of 60 mesh. Black bean, black sesame and perilla were steamed and dried by well known method and ground with granule size of 60 mesh. The above prepared grains, seeds, and extracts of carvacrol were mixed with the ratio of below:

Grains: 30 % (w/w) of brown rice, 15 % (w/w) of Job's tears, 20 % (w/w) of barley, 9 % (w/w) of sweet rice;

Seeds: 7 % (w/w) of perilla, 8 % (w/w) of black bean, 8 % (w/w) of black sesame;

3 % (w/w) carvacrol, 0.5 % (w/w) of Ganoderma lucidum, 0.5 % (w/w) of Rehmannia glutinosa

### Example of Preparation 9

### Chewing Gum

After mixing 20 % (w/w) of gum base, 76.9 % (w/w) of sugar, 1 % (w/w) of flavor 2 % (w/w) of water and 0.1 % (w/w) of extract of Spanish oregano, they were prepared into chewing gum according to a well known method.

### Example of Preparation 10

### Chewing Gum

After mixing 60 % (w/w) of sugar, 39.8 % (w/w) of starch syrup, 0.1 % (w/w) of flavor and extract of Garden savory, they were prepared into candy according to a well known method.

### Example of Preparation 11

### Biscuit

After mixing 25.59 % (w/w) of first grade soft flour, 22.22 % (w/w) of first grade plain flour, 4.80 % (w/w) of refined sugar, 0.73 % (w/w) of salt, 0.78 % (w/w) of glucose, 11.78 % (w/w) of palm shortening, 1.54 % (w/w) of ammonium, 0.17 % (w/w) of Sodium bicarbonate, 0.16 % (w/w) of sodium sulfite, 1.45 % (w/w) rice powder, 0.0001 % (w/w) of vitamin B₁, 0.0001 % (w/w) of vitamin B₂, 0.04 % (w/w) of milk flavor, 20.6998 % (w/w) of water, 1.16 % (w/w) of whole milk powder, 0.29 % (w/w) of alternative milk powder, 0.03 % (w/w) of calcium phosphate I, 0.29 % (w/w) of sulfuric salt, 7.27 % (w/w) of spray milk and 1 % (w/w) of extract of Spanish oregano, they were prepared into biscuit according to a well known method.

### Example of Preparation 12

### Beverage

After mixing 0.26 % (w/w) of honey, 0.0002 % (w/w) of thiotic acid amide, 0.0004

% (w/w) of nicotinic acid amide, 0.0001 % (w/w) of riboflavin natrium HCl, 0.0001 % (w/w) of Pyridoxine HCl, 0.001 % (w/w) inositol, 0.002 % (w/w) ortic acid, 98.7362 % (w/w) of water and 1 % (w/w) of carvacrol, they were prepared into beverage according to a well known method.

### INDUSTRIAL APPLICATION

As described, the present disclosure relates to an use of Spanish oregano (Thymus capitatus) extract, summer savory (Satureja hortensis) extract or carvacrol for treating metabolic diseases, and the present disclosure provides a composition for preventing and treating a metabolic disease selected from the group of obesity, fatty liver, hyperlipidemia and diabetes, comprising Spanish oregano extract, summer savory extract or carvacrol as an active ingredient, an use thereof, and a method of using the same. The composition, use and method according to the present disclosure are useful for weight loss, suppression of abdominal fat accumulation, treatment of fatty liver, reduction of cholesterol and free fatty acid in serum and liver tissue, and improvement of blood glucose level and insulin resistance. Therefore, the composition can be effectively used to prevent and treat a metabolic disease selected from the group consisting of obesity, fatty liver, hyperlipidemia and diabetes.

What is claimed is:

1. A pharmaceutical composition for preventing and treating fatty liver or diabetes (a metabolic disease selected from the group consisting of fatty liver, hyperlipidemia and diabetes) comprising carvacrol represented by Chemical Formula 1 or a pharmaceutically acceptable salt thereof as an active ingredient:

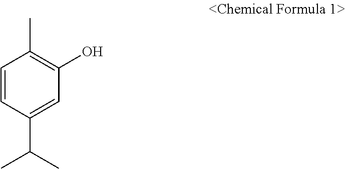

2. A use of carvacrol represented by Chemical Formula 1 or a pharmaceutically acceptable salt thereof for preparing an agent for preventing and treating fatty liver or diabetes (a metabolic disease selected from the group consisting of fatty liver, hyperlipidemia and diabetes).

3. A method for preventing and treating fatty liver or diabetes (a metabolic disease selected from the group consisting of fatty liver, hyperlipidemia and diabetes) comprising administering an effective amount of carvacrol represented by Chemical Formula 1 or a pharmaceutically acceptable salt thereof to a subject in need thereof.l

4. A food composition for preventing and treating fatty liver or diabetes (a metabolic disease selected from the group consisting of fatty liver, hyperlipidemia and diabetes) comprising carvacrol represented by Chemical Formula 1 or a salt thereof as an active ingredient.

5. A pharmaceutical composition for preventing and treating a metabolic disease selected from the group consisting of obesity, fatty liver, hyperlipidemia and diabetes, comprising Spanish oregano (Thymus capitatus) extract or summer savory (Satureja hortensis) extract as an active ingredient.

6. The composition according to claim 5, wherein the Spanish oregano extract or summer savory extract is prepared using water, a C₁-C₆ alcohol or a mixture solvent thereof.

7. An use of Spanish oregano (Thymus capitatus) extract or summer savory (Satureja hortensis) extract for preparing an agent for preventing and treating a metabolic disease selected from the group consisting of obesity, fatty liver, hyperlipidemia and diabetes.

8. The use according to claim 7, wherein the Spanish oregano extract or summer savory extract is prepared using water, a C₁-C₆ alcohol or a mixture solvent thereof.

9. A method for preventing and treating a metabolic disease selected from the group consisting of obesity, fatty liver, hyperlipidemia and diabetes, comprising administering an effective amount of Spanish oregano (Thymus capitatus) extract or summer savory (Satureja hortensis) extract to a subject in need thereof.

10. The method according to claim 9, wherein the Spanish oregano extract or summer savory extract is extracted using water, a C₁-C₆ alcohol or a mixture solvent thereof.

11. A food composition for preventing and treating a metabolic disease selected from the group consisting of obesity, fatty liver, hyperlipidemia and diabetes, comprising Spanish oregano (Thymus capitatus) extract or summer savory (Satureja hortensis) extract as an active ingredient.
